(19) United States
(12) Patent Application Publication
(10) Pub. No.: US 2015/0045751 A1
(43) Pub. Date: Feb. 12, 2015

(54) Excavation Device

(71) Applicant: James Sanders, N. Las Vegas, NV (US)

(72) Inventor: James Sanders, N. Las Vegas, NV (US)

(21) Appl. No.: 13/960,762

(22) Filed: Aug. 6, 2013

(51) Int. Cl. A61M 1/00

(52) U.S. Cl. CPC A61M 1/007
USPC 604/319

(57) ABSTRACT

An excavating device includes a housing that contains a pressure gradient for sucking debris from a body orifice, such as a nose, into the housing. A piston slidably moves along a longitudinal axis of the housing to generate the pressure gradient within the housing. The pressure gradient creates a difference in pressure between a suction end and a pump end of the housing efficacious for sucking debris from a body orifice into the housing. The suction end includes a pair of tubes, such as cannula tubes, operable to at least partially enter the body orifice. Sliding the piston from the suction end to the pump end creates sufficient pressure gradient to suck debris out of the body orifice. A piston rod extends out the pump end of the housing and terminates into a handle for moving the piston. The housing disassembles for cleaning.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to excavation devices. More particularly, the invention relates to a suction pump that generates a pressure gradient to excavate debris from a body orifice.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that an air pump is a device for pushing air. The air may be displaced through a pressure gradient created by the air pump.

Typically, a pressure gradient force is the force which results when there is a difference in pressure across a surface. In general, a pressure is a force per unit area, across a surface. A difference in pressure across a surface then implies a difference in force, which can result in acceleration if there is no additional force to balance it. The resulting force is always directed from the region of higher-pressure to the region of lower-pressure.

It is known that a nose is a protuberance in vertebrates that houses the nostrils, or nares, which admit and expel air for respiration in conjunction with the mouth. Typically, the nose forms mucus. Mucus is a slippery secretion produced by, and covering, mucous membranes. Mucous fluid is typically produced from cells found in mucous glands.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIGS. 1A and 1B illustrate detailed perspective views of an exemplary excavating device, where FIG. 1A illustrates an exemplary excavating device as a complete unit, and FIG. 1B illustrates an exemplary excavating device separated between an exemplary housing and an exemplary piston, in accordance with an embodiment of the present invention;

FIGS. 2A and 2B illustrate detailed perspective views of an exemplary excavating device excavating debris from an exemplary body orifice, where FIG. 2A illustrates an exemplary excavating device at a discharge stroke of the piston, and FIG. 2B illustrates an exemplary excavating device at a suction stroke of the piston, in accordance with an embodiment of the present invention;

FIGS. 3A and 3B illustrate detailed perspective views of an exemplary at least one tube, where FIG. 3A illustrates an exemplary straight at least one tube, and FIG. 3B illustrates an exemplary bent at least one tube, in accordance with an embodiment of the present invention; and

FIG. 4 illustrates a detailed perspective view of an exemplary piston rod and an exemplary handle extending from an exemplary pump end, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

The present invention will now be described in detail with reference to embodiments thereof as illustrated in the accompanying drawings.

There are various types of excavators that may be provided by preferred embodiments of the present invention. In one embodiment of the present invention, the excavating device may include a housing and a piston that slidably engage to generate a pressure gradient within the housing. The pressure gradient creates a difference in pressure between a suction end and a pump end of the housing that serves to suck debris from a body orifice. The suction end may include at least one tube configured to be operable to at least partially enter the body orifice. In some embodiments, sliding the piston along a longitudinal axis of the housing, from the suction end to the pump end, may create sufficient pressure gradient to suck debris out of the body orifice, and into the housing. The at least one tube may act as a bridge between the body orifice and the housing. In some embodiments, the housing may disassemble for accessing and cleaning the debris.

In one embodiment of the present invention, the excavating device may be configured to be operable to at least partially suck debris from body orifices. A housing may provide a sealed chamber for creating the pressure gradient and receiving the debris. The housing may include a cylindrical shape for generating a more efficient pressure gradient. Those skilled in the art, in light of the present teachings, will recognize that the size and dimension of the housing may be manipulated to regulate the pressure gradient, as depicted in the formula:

Force Needed To Create Pressure Gradient=Suction Pressure*Housing Area, or Force=Pressure*Area.

In this manner, changing the velocity of the piston on a suction stroke, or changing the size of the housing may provide greater or lesser pressure gradient forces for sucking the debris.

In some embodiments, the housing may include a suction end. The suction end may include at least one tube, such as cannula tubes, configured to be operable to at least partially enter body orifices. However, in other embodiments, additional types of body orifices may be at least partially entered by the pair of tubes for excavation of debris. The housing may further include a pump end for moving the piston in a reciprocating movement from a suction stroke to a discharge stroke.

In one embodiment of the present invention, the piston may include a disc that forms a snug fit inside the housing, whereby a fluid may not pass around the perimeter of the disc. In this manner, a more efficient pressure gradient may be generated inside the housing. A piston rod may join with the piston, extending out the pump end. A rod cap may pass through the piston rod and form an air tight seal on the open pump end. The pump end may include threads that threadably engage the rod cap to form the seal. A handle may join with the piston rod to provide a grip for operating the piston. In some embodiments, pulling the piston rod away from the suction end—a suction stroke—may remove sufficient fluid from the suction end to create a low pressure relative to the body orifices. Those skilled in the art will recognize that objects flow from a high pressure area to a low pressure area. This directional flow creates suction on the debris from the body orifices to the lower pressured suction end. Conversely, pushing the piston rod towards the suction end prepares the excavation device for another suction stroke to suck debris from the body orifices. In some embodiments the pumping action may be measured to better regulate the force of the pressure gradient.

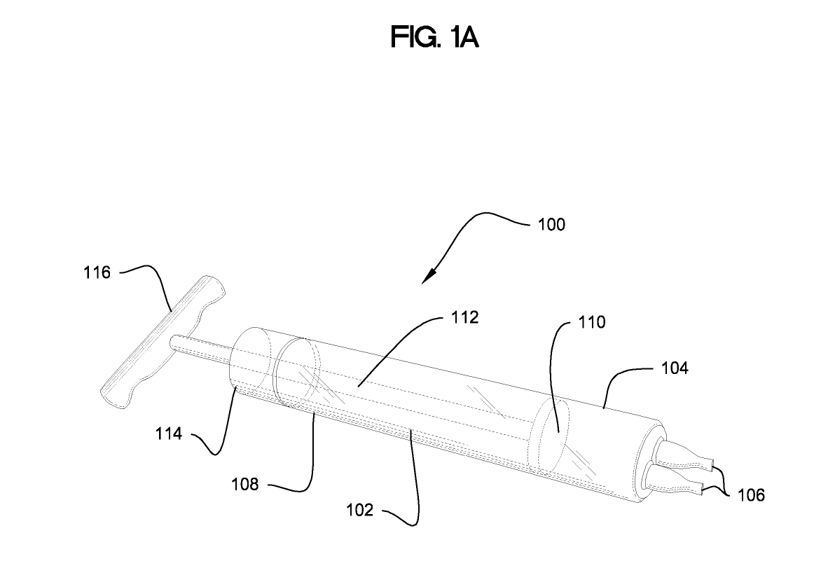

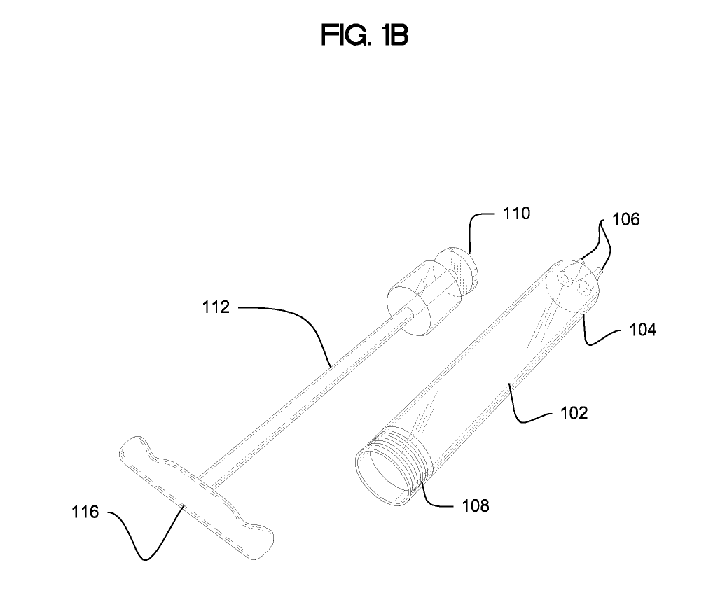

FIGS. 1A and 1B illustrate detailed perspective views of an exemplary excavating device, where FIG. 1A illustrates an exemplary excavating device as a complete unit, and FIG. 1B illustrates an exemplary excavating device separated between an exemplary housing and an exemplary piston, in accordance with an embodiment of the present invention. In the present embodiment, an excavating device 100 may include a housing and a piston that slidably engage to generate a pressure gradient within the housing. The pressure gradient creates a difference in pressure between a suction end and a pump end of the housing efficacious for sucking debris from a body orifice into the housing. The suction end may include at least one tube configured to be operable to at least partially enter the body orifice. In some embodiments, sliding the piston along a longitudinal axis of the housing, from the suction end to the pump end, may create sufficient pressure gradient to suck debris out of the body orifice, and into the housing. The housing may disassemble for accessing and cleaning the debris.

In one embodiment of the present invention, a housing 202 may include a cylindrical shape that is configured to be sufficiently air tight for containing a pressure gradient within. However, in other embodiments, the housing may comprise additional shapes, including, without limitation, a rectangle, a sphere, a triangle, a square, an oval, a pentagon, an octagon, etc. In some embodiments, the housing may include a 5″ length and a ¾″ diameter. The housing may further be transparent to allow a view of the debris. Suitable materials for fabricating the housing may include, without limitation, polyvinyl chloride, polymers, fiberglass, glass, metal, and polyethylene terephthalate The housing may include a suction end 104. The suction end may include at least one tube 106, such as cannula tubes, configured to be operable to at least partially enter body orifices. However, in other embodiments, additional types of body orifices may be at least partially entered by the pair of tubes for excavation of debris. The housing may further include a pump end 108 for moving the piston in a reciprocating movement from a suction stroke to a discharge stroke.

In one embodiment of the present invention, the piston may include a piston 110 that forms a snug fit inside the housing, whereby a fluid may not pass around the perimeter of the piston. The piston may move along a longitudinal axis of the housing to generate the pressure gradient. In some embodiments, pulling the piston from a handle away from the suction end of the housing comprises a suction stroke that at least partially sucks debris from the body orifice. Conversely, pushing the piston towards the suction end comprises a discharge stroke for preparing to excavate additional debris. The piston may include, without limitation, a ¾″ disc shape configured to form a snug fit against an inner area of the housing. It is contemplated that some embodiments may be implemented with pistons of various different shapes such as, but not limited to, square, oval, triangular, pentagonal, octagonal, rectangular, etc. In some instances the use of alternate shapes may reduce the amount of material need in production. For example, without limitation, less material may be needed to make a triangular shape versus a full disc. A rubber seal on the pistons perimeter may enhance the air tightness of the rubber seal. In this manner, a more efficient pressure gradient may be generated inside the housing. A piston rod 112 may join with the piston, extending out the pump end. A rod cap 114 may pass through the piston rod and form an air tight seal on the open pump end. The pump end may include threads that threadably engage the rod cap to form the seal. A handle 116 may join with the piston rod to provide a grip for operating the piston. In some embodiments, pulling the piston rod away from the suction end—a suction stroke—may remove sufficient fluid from the suction end to create a low pressure relative to the body orifices. Those skilled in the art will recognize that objects flow from a high pressure area to a low pressure area. This directional flow creates suction on the debris from the body orifices to the lower pressured suction end. Conversely, pushing the piston rod towards the suction end prepares the excavation device for another suction stroke to suck debris from the body orifices.

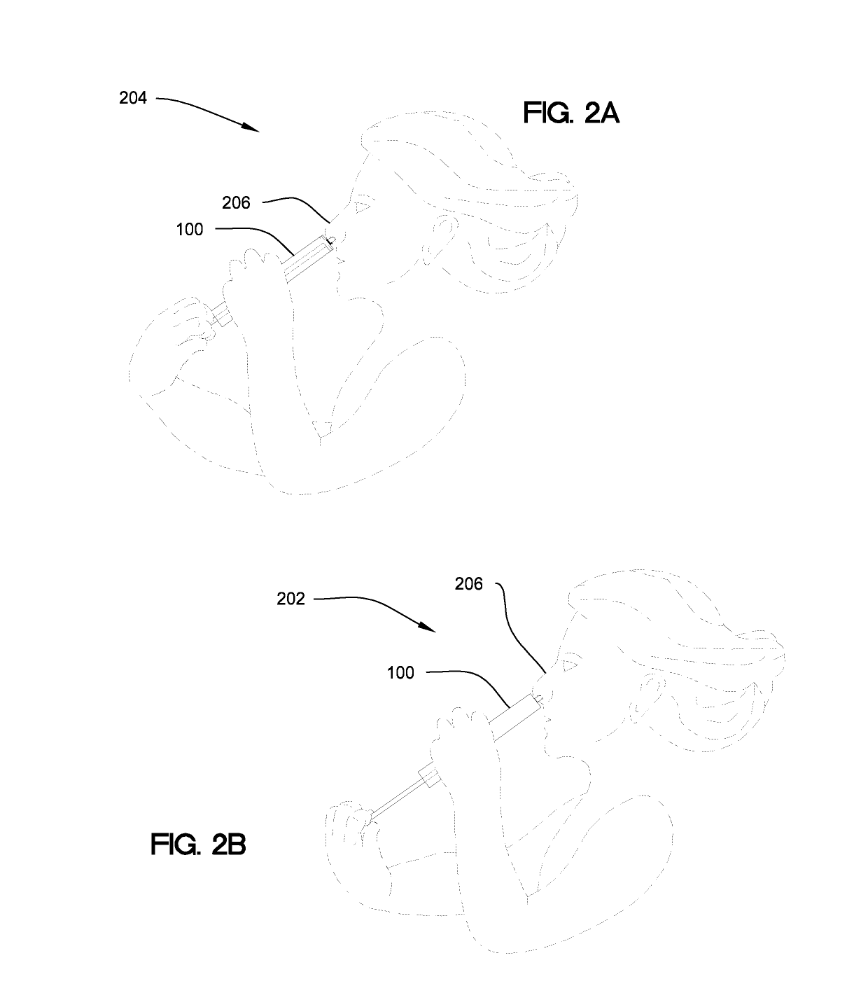

FIGS. 2A and 2B illustrate detailed perspective views of an exemplary excavating device excavating debris from an exemplary body orifice, where FIG. 2A illustrates an exemplary excavating device at a discharge stroke of the piston, and FIG. 2B illustrates an exemplary excavating device at a suction stroke of the piston, in accordance with an embodiment of the present invention. In the present embodiment, the excavating device may be configured to be operable to at least partially suck debris from a body orifice 206. The debris may include, without limitation, mucus, dust, germs, and allergens. A housing may provide a sealed chamber for creating the pressure gradient and receiving the debris. The housing may include a cylindrical shape for generating a more efficient pressure gradient. In some embodiments, pulling the piston from a handle away from the suction end of the housing comprises a suction stroke 202 that at least partially sucks debris from the body orifice. Conversely, pushing the piston towards the suction end comprises a discharge stroke 204 for preparing to excavate additional debris. Those skilled in the art, in light of the present teachings, will recognize that the size and dimension of the housing may be manipulated to regulate the pressure gradient, as depicted in the formula:

Force Needed To Create Pressure Gradient=Suction Pressure*Housing Area, or Force=Pressure*Area.

In one embodiment, the force may include the amount required to pull the piston into a suction stroke with sufficient pressure gradient generated to suck debris from the body orifice. The pressure may include the pressure gradient. The area may include the volume of the housing, often having a cylindrical shape. In this manner, changing the velocity of the piston on a suction stroke, or changing the size of the housing may provide greater or lesser pressure gradient forces for sucking the debris.

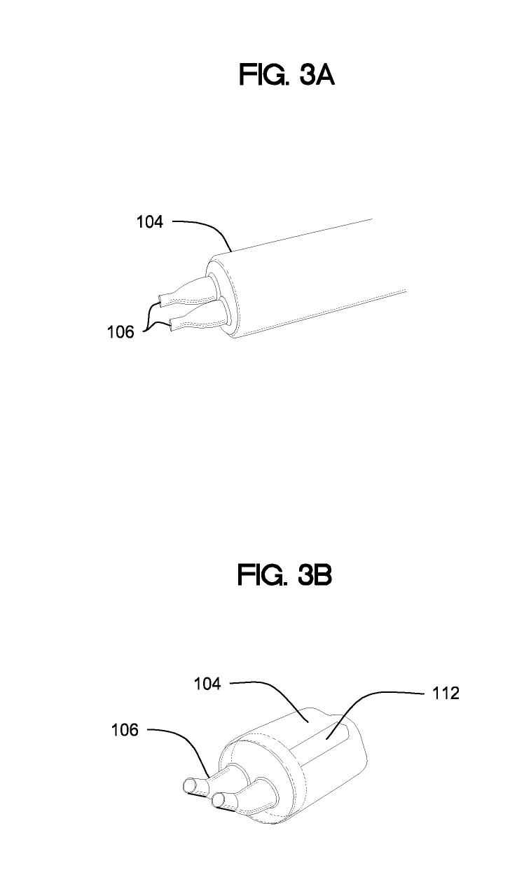

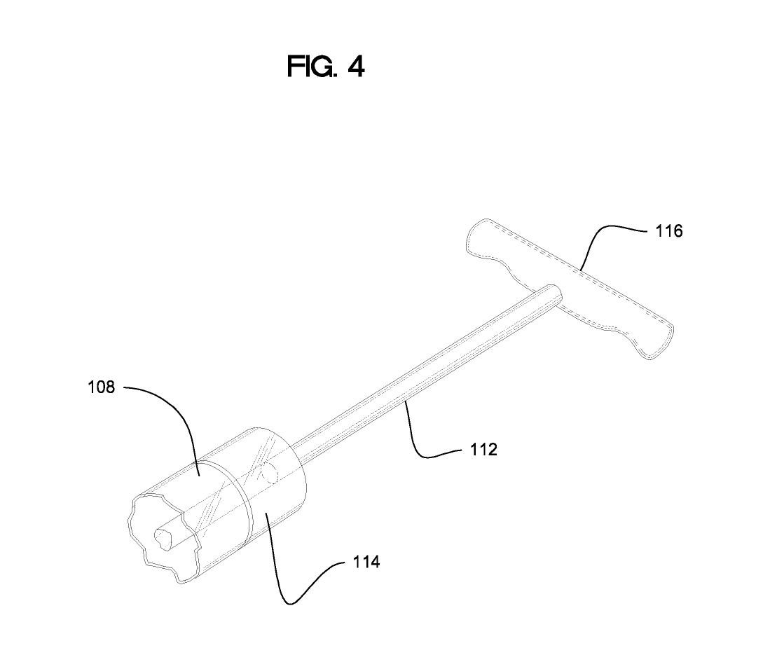

FIGS. 3A and 3B illustrate detailed perspective views of an exemplary at least one tube, where FIG. 3A illustrates an exemplary straight at least one tube, and FIG. 3B illustrates an exemplary bent at least one tube, in accordance with an embodiment of the present invention. In the present embodiment, the suction end may include at least one tube configured to be operable to at least partially enter body orifices. The tube may include, without limitation, a cannula tube. The at least one tube may include a pair of bifurcated cannula tubes having a tapered shape for facilitated entry into a pair of nostrils. The pair cannula tubes may be ½″ long. The at least one tube may be sufficiently flexible to bend and conform to the body orifice. Suitable materials for the tubes may include, without limitation, rubber, malleable polymers, silicone, and plastic. However, in other embodiments, additional types of body orifices may be at least partially entered by the pair of tubes for excavation of debris, including, without limitation, ears, mouth, throat, and sexual organs. The at least one tube may detach for cleansing.

FIG. 4 illustrates a detailed perspective view of an exemplary piston rod and an exemplary handle extending from an exemplary pump end, in accordance with an embodiment of the present invention. In the present embodiment, a handle may join with the piston rod to provide a grip for operating the piston. The handle may be a part of, or attachment to, the piston rod configured to be operable to be moved or used by hand. The handle may include a sufficiently small circumference to permit the hand to surround the handle far enough to grip it as solidly as needed to exert a sufficient force to generate the pressure gradient in the housing. In one embodiment, the handle may include a plunger type handle having finger-grip indentations.

In one alternative embodiment, the excavation device may include an automated motor pump rather than a manual hand pump to generate the pressure gradient. In yet another alternative embodiment, the excavation device is used in surgical opening to clean out blood clots and other surgical contaminants. In yet another alternative embodiment, the at least one tube may include interchangeable cannula tubes for fitting into differently sized nostrils, or to engage different body orifices. In yet another alternative embodiment, the housing may include a solution to kill germs and bacteria. In yet another alternative embodiment, the housing may fasten around the neck of a user such that both hands can be used to pump the piston.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing a manual pump and a pair of tubes to excavate debris from a body orifice according to the present invention will be apparent to those skilled in the art. Various aspects of the invention have been described above by way of illustration, and the specific embodiments disclosed are not intended to limit the invention to the particular forms disclosed. The particular implementation of the manual pump and a pair of tubes to excavate debris from a body orifice may vary depending upon the particular context or application. By way of example, and not limitation, the manual pump and a pair of tubes to excavate debris from a body orifice described in the foregoing were principally directed to a hand pump that joins with a pair of cannula tubes to extract mucus from the nose implementations; however, similar techniques may instead be applied to excavating debris from ears through the same pressure gradient pump, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A device comprising:
a housing, said housing being configured to contain a pressure gradient, said pressure gradient being configured to be operable to at least partially suck debris from a body orifice, said housing comprising a suction end, said suction end comprising at least one tube, said at least one tube being configured to be operable to at least partially enter said body orifice, said at least one tube being operable to at least partially carry said debris from said body orifice to said suction end in response to said pressure gradient, said housing further comprising a pump end, said pump end being configured to control the formation of said pressure gradient; and
a piston, said piston being disposed inside said housing, said piston being configured to move along a longitudinal axis of an inner perimeter of said housing, said piston being operable to move from said suction end to said pump end to generate said pressure gradient.

2. The device of claim 1, in which said body orifice comprises a nose.

3. The device of claim 1, in which said debris comprises mucus.

4. The device of claim 1, in which said housing comprises a cylindrical shape having a 5 inch length and a ¾inch circumference.

5. The device of claim 1, wherein said housing forms an air tight seal with said piston.

6. The device of claim 1, in wherein said housing is at least partially transparent.

7. The device of claim 1, in which said at least one tube comprises a ½ inch long cannula having a substantially tapered shaped.

8. The device of claim 1, in which said at least one tube comprises a pair of tubes configured to at least partially enter a pair of nostrils.

9. The device of claim 1, wherein said at least one tube is sufficiently flexible to form a seal with said body orifice.

10. The device of claim 1, in which said piston comprises a disc shape.

11. The device of claim 1, wherein a piston circumference is larger than a housing circumference, said piston further comprising a rubber seal perimeter to form a tight fit with an inner surface of said housing.

12. The device of claim 1, in which said piston comprises a piston rod.

13. The device of claim 12, wherein said piston rod is configured to help move said piston along said longitudinal axis of said housing.

14. The device of claim 13, wherein said piston rod is disposed to join with said piston at a rod first end, and extend from said pump end from a rod second end.

15. The device of claim 14, in which said rod second end comprises a handle for gripping and operating said piston rod.

16. The device of claim 15, in which said piston rod comprises a rod cap.

17. The device of claim 16, wherein said rod cap is disposed to pass through said piston rod and at least partially cover said pump end.

18. The device of claim 17, wherein said rod cap threadably engages said pump end to form an airtight seal.

19. A device comprising:
means for orienting at least one tube to face a body orifice;
means for at least partially entering said body orifice with said at least one tube;
means for pulling a piston away from a suction end towards a pump end of a housing;
means for generating a pressure gradient inside said housing;
means for sucking a debris from said body orifice into said housing; and
means for pushing said piston from a pump end towards a suction end to initiate additional excavation of said body orifice.

20. A device consisting of:
a housing, said housing comprising a cylindrical shape, said housing being configured to contain a pressure gradient, said pressure gradient being configured to be operable to at least partially suck debris from a body orifice, said debris comprising mucus, said body orifice comprising a nose, said housing comprising a suction end, said suction end comprising at least one tube, said at least one tube comprising a pair of cannula tubes, said pair of cannula tubes being tapered, said at least one tube being configured to be operable to at least partially enter said body orifice, said at least one tube being operable to at least partially carry said debris from said body orifice to said suction end in response to said pressure gradient, said housing further comprising a pump end, said pump end being configured to control the formation of said pressure gradient; and
a piston, said piston being disposed inside said housing, said piston being configured to move along a longitudinal axis of an inner perimeter of said housing, said piston being operable to move from said suction end to said pump end to generate said pressure gradient, said piston having a piston circumference greater than a housing circumference, said piston comprising a perimeter rubber seal for forming a tight fit with a housing inner area, said piston comprising a piston rod, said piston rod being configured to help move said piston along a longitudinal axis of said housing, said piston rod being disposed to join with said piston at a rod first end and extend from said pump end from a rod second end, said rod second end comprising a handle for gripping and operating said piston rod, said piston rod further comprising a rod cap, said rod cap being disposed to pass through said piston rod and at least partially cover said pump end, said rod cap being configured to threadably engage said pump end to form an airtight seal.
